(19) United States
(12) Patent Application Publication
(10) Pub. No.: US 2023/0255638 A1
(43) Pub. Date: Aug. 17, 2023

(54) DEVICE FOR ASSISTING IN THE PLACEMENT OF AN UMBILICAL CATHETER IN PREMATURE AND NEWBORN INFANTS

(71) Applicant: pfm medical ag, Koln (DE)

(72) Inventors: Pia Welfers, Wegberg (DE); Bianca Haase, Tubingen (DE); Georg Kox, Wegberg (DE); Daniel Rauch, Essen (DE)

(21) Appl. No.: 18/169,521

(22) Filed: Feb. 15, 2023

(30) Foreign Application Priority Data
Feb. 16, 2022 (IT) 102022000002873

(51) Int. Cl. A61B 17/122

(52) U.S. Cl. CPC A61B 17/1227

(57) ABSTRACT

A device for assisting in the placement of an umbilical catheter in premature and newborn infants comprises a first leg and a second leg. The legs each comprise a semi-cylindrical recess in the region of a first end and are connected to one another in the region of the second end via a spring element. The spring element holds the legs by a spring force in a first operating state in which the respective first ends of the legs are spaced apart from each other. The legs can be transferred against the spring force into a second operating state in which the respective first ends of the legs are arranged directly adjacent to each other. The semi-cylindrical recesses in the region of the respective first end of the legs together form a cylindrical receptacle for the umbilical cord of the infant in the second operating state.

### RELATED APPLICATIONS AND CLAIM OF PRIORITY

This patent document claims priority to Italy patent application number IT 102022000002873, filed Feb. 16, 2022.

### BACKGROUND

The patent document relates to a device for assisting in the placement of an umbilical catheter in premature and newborn infants, in particular into an umbilical cord vessel of a premature and newborn infant.

During the initial care of a premature or newborn infant, vascular access may be needed (immediately) to administer medications or fluids to the premature or newborn infant. It is of particular importance that the initial care of the premature and newborn infant in the context of resuscitation must be particularly fast and safe.

In the state of the art, three different methods of vascular access are known and regularly used: the umbilical catheter, peripheral venous access and intraosseous access (i.o. access). All accesses are placed by experienced personnel. This requires a great deal of practice and experience on the part of the medical personnel. Particularly in the case of the first alternative, several people are busy placing the vascular access at the same time. This often leads to space problems around the premature and newborn infant.

The rapid placement of a peripheral venous catheter (PVK) is performed by specially trained and experienced personnel. However, the placement is not always successful and is all the more difficult the more immature or depressed the premature or newborn infant is. Especially during resuscitation, the insertion of the catheter is often particularly difficult. Possible puncture sites are the dorsum of the hand, the dorsum of the foot, the veins of the head and the cubital veins. Especially in very small premature infants and premature and newborn infants in a poor circulatory situation, the placement of peripheral venous access is usually not possible.

As an alternative access, an i.o. approach can be made via the proximal tibia. However, the i.o. approach is only permissible from a weight of 3000 grams and carries additional risks, such as bone injuries, inflammations and growth disturbances of the lower leg bone. However, at present it is technically easier to insert an i.o. access than an umbilical catheter and it is therefore often much faster.

Another alternative access is the umbilical catheter. In this case, a blood vessel of the umbilical cord is provided with a catheter or cannulated. The blood vessel may be an umbilical vein or an umbilical artery. Venous or arterial access in the first minutes of life in the critically ill premature and newborn infant is essential to administer volume boluses, glucose, and emergency medications such as Suprarenin. Failure to do so is likely to result in sequelae of hypotension, hypoglycemia, or persistent bradycardia.

The main problem when placing an umbilical vein catheter (UVC) or umbilical artery catheter (UAC) is to detect the blood vessels and to prepare them in such a way that the vessel lumen becomes visible. In the current state of the art, this is solved, for example, by manually spreading the umbilicus using two surgical forceps. This requires at least one other person to present the umbilicus in such a way that a second person can place the catheter.

Thus, there are two major disadvantages to placing an umbilical catheter. First, it requires at least two experienced physicians who are coordinated to place a catheter. These physicians both work actively on the umbilicus, so that space problems can arise around the premature and newborn infant. Secondly, it requires a lot of experience of the attending physicians. Placing a catheter usually turns out to be very difficult in terms of craftsmanship or ends frustratingly.

To overcome these two disadvantages, WO 2018/202844 A1 discloses a medical device for placing a catheter into an umbilical cord blood vessel of a premature infant, the medical device having a longitudinal axis, a proximal end, and a distal end. The proposed device is configured as a sheath to enclose a portion of an umbilical cord. The medical device includes a clamping device to clamp the umbilical cord, a cutting device to cut the umbilical cord to form an umbilical interface, and a hooking device to expand the umbilical interface. The medical device clamps the umbilical cord, separates the umbilical cord, and spreads the umbilical cord open for placement of the umbilical catheter. However, a disadvantage of the medical device is its complex structure and associated manufacturing costs. Although the medical device is reusable, it must be disinfected after each use, which is costly, especially because of the large number of moving elements. Another disadvantage is that the position and anatomical shape of the umbilical vessels varies greatly and therefore the solution described in the above patent can hardly be implemented.

### SUMMARY

This document discloses various embodiments of a new medical device for placing a catheter into an umbilical cord blood vessel, which simplifies the difficult insertion of a catheter via an umbilical cord blood vessel and avoids the disadvantages of medical devices known in the prior art.

In various embodiments, the problem is solved by a device for assisting in the placement of an umbilical catheter in premature and newborn infants, comprising: a first leg and a second leg, wherein the first and second legs each comprise a semi-cylindrical recess in the region of a first end and are connected to one another in the region of the second end via a spring element, wherein the spring element holds the first and second leg by a spring force in a first operating state in which the respective first ends of the first and second leg are spaced apart from each other, and wherein the first and second legs can be transferred against the spring force to a second operating state in which the respective first ends of the first and second leg are arranged directly adjacent to each other, wherein the semi-cylindrical recesses in the region of the respective first end of the first and second legs together form a cylindrical receptacle for the umbilical cord of the premature and newborn infant in the second operating state.

Some variants of the invention, the device comprises a locking device for locking the device in the second operating state. This means that the user of the device does not have to hold it in the second operating state against the spring force, but the device is locked in this second operating state. The user can thus fully concentrate on cutting the umbilical cord and placing the umbilical catheter. Preferably, the locking device locks automatically when the user has transferred the device to the second operating state.

According to some variants of the invention, the locking device is designed to be detachable, in particular as a latching connection, in order to be able to remove the device again after the umbilical catheter has been placed. For this purpose, for example, the latching connection is released by moving the latch.

Pursuant to some variants, the locking device is formed at the first end of the first and second leg, in particular a first part of the locking device is formed at the first end of the first leg and a second part of the locking device is formed at the first end of the second leg. Thus, in particular, the first ends of the first and second legs may be securely fixed relative to each other by the locking device, precisely in the region in which the cylindrical receptacle for the umbilical cord is formed in the second operating state. The umbilical cord is therefore securely fixed in the receptacle, as the first ends of the first and second legs cannot move due to the locking device. For example, the first end of the first leg may have an eyelet, and the first end of the second leg may have a detent (protrusion) that engages the eyelet in the second operating state.

In some variants, the device comprises an elevation on the distal side of the device in the region of the cylindrical receptacle of the umbilical cord in the second operating state. This elevation serves as a cutting edge (counter-bearing) when cutting the umbilical cord. The umbilical cord is cut by means of a scalpel which is guided along the distal edge of the device. The elevation is arranged behind the umbilical cord in the cutting direction so that the umbilical cord is supported by the elevation during cutting, making it easier to cut.

According to some variants, the elevation is divided into two parts and one part of the elevation is arranged on each of the first and second legs. In the second operating state, the two parts of the elevation are arranged immediately adjacent to each other. Typically, a cut is always directed away from the user's body for safety reasons. In this case, that means the cut to cut through the umbilical cord is made toward the first end of the device. The elevation must therefore be located at the first end of the device, such as in the cutting direction after the cylindrical receptacle for the umbilical cord. In this area, however, the first and second legs are separated from each other in the first operating state so that the umbilical cord can be received in the device. For this reason, it is expedient if each leg has a portion of the elevation, which together form the elevation in the second operating state.

In some variants, a semi-circular element is arranged at the distal end of the respective portion of the elevation, said semi-circular element being axially aligned with the semi-cylindrical recess of the respective first and second leg, said two semi-circular elements forming a circular receptacle in the second operating state, to secure the umbilical cord of the premature and newborn infant, the circular receptacle being axially aligned with and spaced from the cylindrical receptacle in the second operating state so as to leave a cut channel between the cylindrical receptacle and the circular receptacle. As a result, the umbilical cord of the premature infant is fixed both below the cutting channel by the cylindrical receptacle and above the cutting channel by the circular receptacle. This makes it particularly easy to cut the umbilical cord.

According to some variants, the elevation or the parts of the elevation are connected to the first and/or second leg of the device via a predetermined breaking edge. The elevation can thus be easily separated from the device after the umbilical cord has been cut by simply snapping it off. This prevents the elevation or parts connected thereto, such as the semicircular elements, from obstructing the insertion of the catheter into a vessel of the umbilical cord.

Pursuant to a further variant, the semi-cylindrical recesses in the first operating state and the cylindrical receptacle in the second operating state are conical in shape, the cone widening from the proximal side to the distal side of the device. As a result, the umbilical cord may be particularly well fixed in the cylindrical receptacle. Furthermore, this allows, for example, the vessels in the umbilical cord to be squeezed in the proximal region, i.e. the narrower region of the cone, so that no or only very little blood can escape from the vessels of the umbilical cord after cutting. The cone may be designed in such a way that the catheter can be inserted sufficiently deep into the vessel of the umbilical cord to provide access to the vascular system of the premature and newborn infant.

According to some variants, the semi-cylindrical recesses in the first operating state and the cylindrical receptacle in the second operating state are grooved on the inside, in particular have openings to form lamellae. This further improves the fixation of the umbilical cord in the cylindrical receptacle.

In some variants, the first and second legs each have outwardly extending wings on the distal side as finger guards. These protect the fingers of the user of the device from cuts when cutting the umbilical cord.

Pursuant to some variants, the spring element is circular in shape, which is open in the first operating state and closed in the second operating state. In this way, the spring element can be easily manufactured and integrated into the device. Due to the diameter of the circular spring element, the spring force can be adjusted in advance.

According to some variants, the device may be formed in one piece. A one-piece device has the advantage that it can be easily disinfected. In particular, such simply one-piece devices can be manufactured inexpensively, so that they may be designed as a disposable product, since the manufacturing is less expensive than disinfecting the device.

Optionally, the device according to the invention is made of a plastic.

For better handling, the first and second legs can have gripping surfaces which are grooved, for example. This improves the grip.

Optionally, the devices are manufactured and stocked with different diameters of the cylindrical receptacle in the second operating state so that, in an emergency, a device with the appropriate diameter for the current umbilical cord can be selected quickly. For example, the cylindrical receptacle in the second operating state may have a diameter of 5 mm to 15 mm, or 7 mm, 9 mm or 11 mm.

### BRIEF DESCRIPTION OF THE DRAWINGS

The invention is explained in more detail below with reference to examples of embodiments shown in the figures.

FIG. 1 shows a perspective view of an embodiment of a device for assisting in the placement of an umbilical catheter in premature and newborn infants in the first operating state,

FIG. 2 shows a top view of the device from FIG. 1,

FIG. 3 shows a perspective view of an embodiment of the device in the second operating state with fixed umbilical cord before cutting,

FIG. 4 shows a perspective view of the device of FIG. 3 after cutting the umbilical cord and removing the elevation, and

FIG. 5 shows an umbilical catheter placed after removal of the device.

### DETAILED DESCRIPTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” (or “comprises”) means “including (or includes), but not limited to.” When used in this document, the term “exemplary” is intended to mean “by way of example” and is not intended to indicate that a particular exemplary item is preferred or required.

In this document, when terms such as “first” and “second” are used to modify a noun, such use is simply intended to distinguish one item from another, and is not intended to require a sequential order unless specifically stated. The term “approximately,” when used in connection with a numeric value, is intended to include values that are close to, but not exactly, the number. For example, in some embodiments, the term “approximately” may include values that are within +/−10 percent of the value.

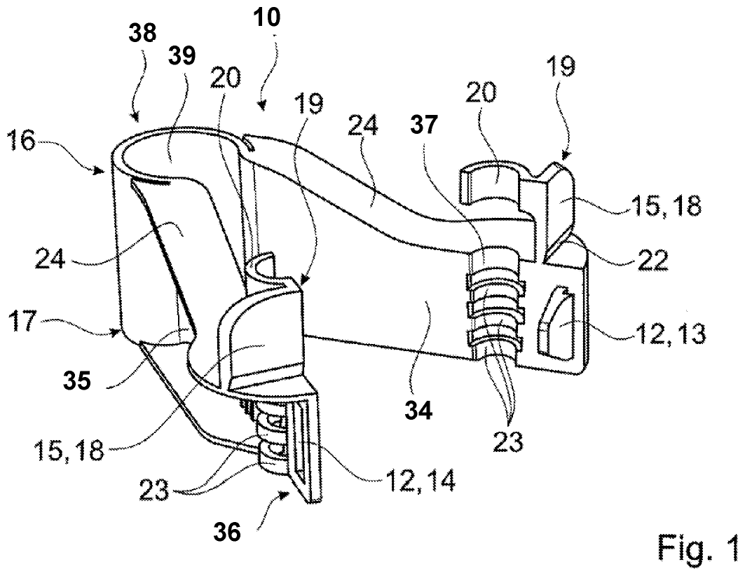

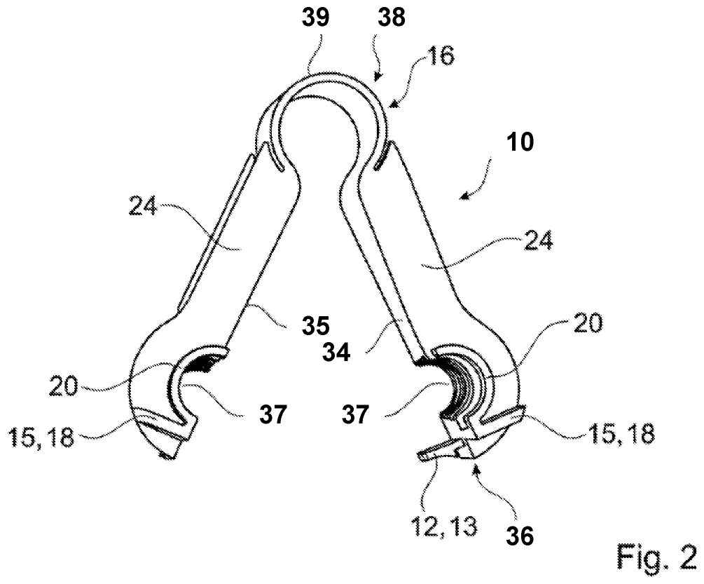

FIG. 1 shows a perspective view of an embodiment of a device 10 for assisting in the placement of an umbilical catheter 32 in premature and newborn infants 33. FIG. 2 shows a top view of the device 10 of FIG. 1. The device 10 comprises a first leg 34 and a second leg 35. The first leg 34 and the second leg 35 each have a semi-cylindrical recess 37 in the region of a first end 36. In the region of a second end 38, the first leg 34 and the second leg 35 are connected to one another via a spring element 39.

The spring element 39 holds the first leg 34 and the second leg 35 by an inherent spring force in a first operating state, in which the respective first ends 36 of the first leg 34 and the second leg 35 are spaced apart. FIG. 1 shows the device 31 in the first operating state.

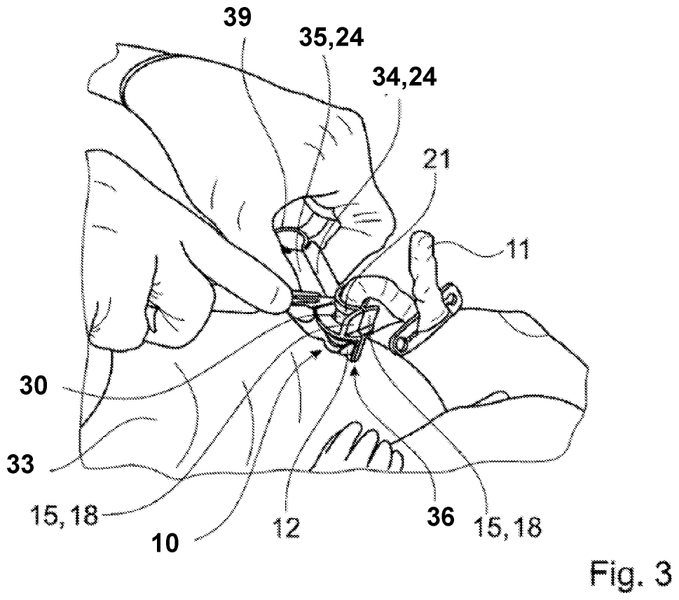

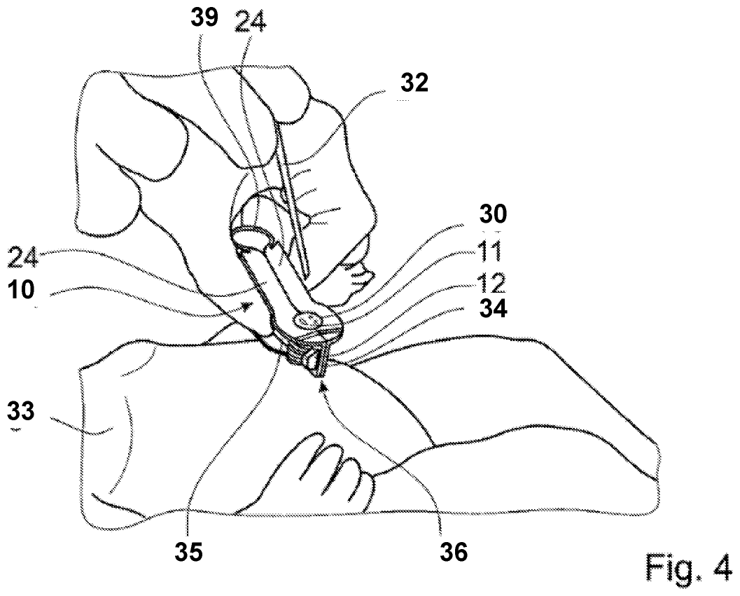

The first leg 34 and the second leg 35 can be transferred into a second operating state against the spring force, in which the respective first ends 36 of the first leg 34 and the second leg 35 are arranged directly adjacent to each other. FIGS. 3 and 4 show, for example, a device 10 in the second operating state.

According to the first embodiment of FIG. 1, the device 10 comprises a locking device 12, for locking the device 10 in the second operating state. In the first embodiment, the locking device 12 is designed as a releasable latching connection. The locking device designed as a snap-in connection is arranged at the first end 36 of the first leg 34 and second leg 35, wherein a first part 13 of the locking device 12 is arranged at the first end 36 of the first leg 34 and a second part 14 of the locking device 12 is arranged at the first end 36 of the second leg 35. According to the illustrated embodiment, a projection (latch) is formed as a first part 13 at the first end 36 of the first leg 34, which projection can engage in an opening (eyelet) at the first end 36 of the second leg 35, the opening forming the second part 14 of the locking device 12. In a second embodiment, the locking device 12 may provide a fixed, non-releasable connection.

The device 10 of FIG. 1 further comprises an elevation 15 on the distal side 16 of the device 10 in the region of the cylindrical receptacle 30 in the second operating state. The distal side in the sense of this disclosure is thereby the side of the device 10 facing away from the premature and newborn infant 33, and the proximal side is the side of the device 10 facing the premature and newborn infant 33.

The elevation 15 is arranged in the region of the cylindrical receptacle 30 in the second operating state or of the semi-cylindrical recesses 17 in the first operating state. According to the first embodiment shown in FIG. 1, the elevation is formed in two parts, with one part of the elevation 18 being arranged on each of the first and second leg 34, 35. In the second operating state of the device 10, the two parts 18 of the elevation 15 are arranged directly adjacent to each other.

A semicircular element 20 is disposed at the distal end 19 of the respective portion 18 of the elevation 15. The semicircular element 20 is axially aligned with the semicylindrical recess 17 of the respective first and second leg 14, 15. In the second operating state, the two semicircular elements 20 form a circular receptacle 21 to fix the umbilical cord 11 of the premature and newborn infant 13. In the second operating state, the circular receptacle 21 is axially aligned with and spaced from the cylindrical receptacle 30 so as to leave a cutting channel between the cylindrical receptacle 30 and the circular receptacle 21.

Optionally, the elevation 15 or parts of the elevation 18 may be connected to the first and/or second leg 14, 15 of the device 10 via a predetermined breaking edge 22. This allows the elevation 15 to be quickly and easily removed from the device 10 by buckling so that it does not obstruct the insertion of the umbilical catheter 12 into a vessel of the umbilical cord 11.

Advantageously, the semi-cylindrical recesses 17 in the first operating state and the cylindrical receptacle 30 in the second operating state may be conical in shape, the cone widening from the proximal side 17 to the distal side 16 of the device 10. As a result, the umbilical cord 11 is particularly well fixed in the device 10. In particular, the vessels of the umbilical cord 11 can be squeezed in this way so that no blood escapes after cutting, but at the same time the umbilical catheter 12 can be inserted into the vessel of the umbilical cord 11.

To further improve the fixation of the umbilical cord 11 in the cylindrical receptacle 30 of the device 10 in the second operating state, the semi-cylindrical recesses 17 or the cylindrical receptacle 30 have perforations, for forming lamellae 23.

Furthermore, the first leg 41 and the second leg 15 each may have an outwardly extending wing 24 on the distal side 16 as a finger guard.

As can be seen in particular from FIG. 2, the spring element 19 is circular, which is open in the first operating state. As can be seen from FIG. 3, the circular spring element 19 is closed in the second operating state.

Optionally the device 10 may be formed in one piece and/or made of a plastic. This enables cost-effective manufacture, so that the device 10 can be offered in particular as a disposable product.

FIGS. 3 to 5 show the use of a device 10 for placing an umbilical catheter 12 in premature and newborn infants 13. In the first operating state of the device 10, the umbilical cord 11 of the premature and newborn infant 13 is placed between the first leg 14 and the second leg 15 in the area of the two semi-cylindrical recesses 17. Thereafter, the device 10 is transferred to the second operating state by the user moving the first leg 14 and the second leg 15 of the device 10 towards each other. Once the first and second legs 14, 15 are immediately adjacent to each other, the device 10 is fixed in this second operating state by the locking device 12. FIG. 3 shows this second operating state.

The user can now cut the umbilical cord 11. To do this, the scalpel 25 is guided along the distal side of the device 10, with the distal edge of the device 10 serving as a guide. The umbilical cord 11 is fixed in place by the circular receptacle 21 and the cylindrical receptacle 30. The umbilical cord 11 is cut against the elevation 15.

Subsequently, the elevation 15 is bent so that the cut umbilical cord 11, in particular its vessels, is clearly visible and accessible. This condition is shown in FIG. 4. The umbilical catheter 12 can now be quickly and easily inserted into the desired vessel of the umbilical cord 11.

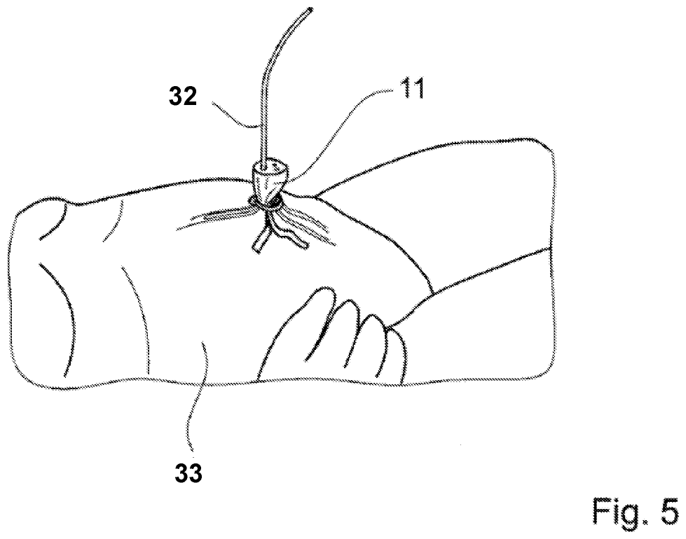

As shown in FIG. 5, after the umbilical catheter 12 has been placed, the device 10 may be removed by releasing the locking device 12. To prevent blood leakage from the non-catheterized vessels of the umbilical cord, the latter is tied. Care should be taken to ensure that the placed umbilical catheter 12 remains open.

The device described above has a particularly simple design, since only two movable legs are connected to each other via a spring element. The umbilical cord may be fixed in the cylindrical receptacle in the second operating state. The distal edges of the first and second legs, which are adjacent to each other in the second operating state, serve as a guide for the scalpel to cut the umbilical cord at the distal edge of the device. In this way, a particularly straight cutting edge is achieved and it has been found that with such a cutting edge, the spreading of the umbilical cord required in the prior art can be avoided, since the vessels in the umbilical cord can be clearly seen and the catheter can also be inserted without any problems.

By means of the device described above, an umbilical catheter can be easily and quickly inserted into the vessels of the umbilical cord of a premature and newborn infant. For this purpose, the umbilical cord is arranged between the first and second leg of the device in the first operating state in such a way that it is arranged in the receptacle at the first end of the first and second legs when compressed and thereby transferred to the second operating state. Once the umbilical cord is fixed in the formed cylindrical receptacle in the second operating state, it can be cut with a scalpel, the scalpel being guided along the distal edge of the device according to the invention, thereby obtaining a clean cut. Subsequently, the catheter can be inserted into the desired vessel of the umbilical cord of the premature and newborn infant. If the device is now released, it automatically assumes the first operating state in which the first leg and the second leg are spread apart so that the umbilical cord is released. To prevent blood from leaking from the non-catheterized vessels, the umbilical cord can be tied off. This simultaneously fixes the catheter in the clamped vessel so that it can no longer be displaced.

The device has a particular simple design, particularly without moving parts such as swiveling knives or hooks. As a result, the device can be sterilized very easily. Alternatively, however, the device can also be manufactured without difficulty as a disposable product and disposed after use, since the simple structure permits inexpensive manufacture. The disadvantages of the prior art are thus avoided.

The features and functions described above, as well as alternatives, may be combined into many other different systems or applications. Various alternatives, modifications, variations or improvements may be made by those skilled in the art, each of which is also intended to be encompassed by the disclosed embodiments.

1. A device for assisting in the placement of an umbilical catheter in premature and newborn infants, comprising:
a first leg and a second leg, each of which comprises a first end and a second end,
wherein the first and second leg each comprise a semi-cylindrical recess in a region of the first ends of each leg and are connected to one another in a region of the second ends of each leg via a spring element,
wherein the spring element holds the first and second legs by a spring force in a first operating state in which the respective first ends of the first and second leg are spaced apart from each other and wherein the first and second leg can be transferred against the spring force into a second operating state in which the respective first ends of the first and second leg are arranged directly adjacent to each other,
wherein the semi-cylindrical recesses in the region of the first end of the first and second leg together form a cylindrical receptacle for an umbilical cord of an infant in the second operating state.

2. The device according to claim 1, further comprising:
a locking device, for locking the device in the second operating state.

3. The device according to claim 2,
wherein the locking device is releasable and comprises a snap-in connection.

4. The device according to claim 2,
wherein the locking device is non-releasable.

5. The device according to claim 2,
wherein a first part of the locking device is formed at the first end of the first leg and a second part of the locking device is formed at the first end of the second leg.

6. The device according to claim 1, further comprising:
an elevation on a distal side of the device in a region of the cylindrical receptacle when the device is in the second operating state.

7. The device according to claim 6,
wherein the elevation comprises two parts and a part of the elevation is arranged on each of the first and second legs, the two parts of the elevation being immediately adjacent to each other when the device is in the second operating state.

8. The device according to claim 7,
wherein at a distal end of the elevation two semicircular elements are arranged, which are axially aligned with the semi-cylindrical recesses of the first and second legs, wherein the two semicircular elements form a circular receptacle when the device is in the second operating state, to fix the umbilical cord of the infant, the circular receptacle being axially aligned with and spaced from the cylindrical receptacle when the device is in the second operating state, so that a cutting channel remains between the cylindrical receptacle and the circular receptacle.

9. The device according to any claim 6,
wherein the elevation is connected to the first and/or second leg of the device via a predetermined breaking edge.

10. The device according to claim 1,
wherein the semi-cylindrical recesses are conical in the first operating state and the cylindrical receptacle is conical in the second operating state, the cone widening from the proximal side to the distal side of the device.

11. The device according to claim 1, wherein
when the device is in the first operating state the semi-cylindrical recesses are grooved and has openings for forming lamellae; and
when the device is in the second operating state the cylindrical receptacle is grooved and has openings for forming lamellae.

12. The device according to claim 1,
wherein the first and second legs each have outwardly extending wings on a distal side as finger protection.

13. The device according to claim 1,
wherein the spring element is circular, is open when the device is in the first operating state, and is closed when the device is in the second operating state.

14. The device according to claim 1,
wherein the device is formed in one piece.

15. The device according to claim 1,
wherein the device is made of a plastic material.
